(19) United States
(12) Patent Application Publication
(10) Pub. No.: US 2015/0168297 A1
(43) Pub. Date: Jun. 18, 2015

(54) System to Distinguish between X Sperm and Y Sperm

(71) Applicants: Satish Deshpande, Guelph (CA); Donald F. Moyer, Chicago, IL (US); Bethany Deshpande, Quebec City (CA)

(72) Inventors: Satish Deshpande, Guelph (CA); Donald F. Moyer, Chicago, IL (US); Bethany Deshpande, Quebec City (CA)

(21) Appl. No.: 14/106,837

(22) Filed: Dec. 15, 2013

(51) Int. Cl. G01N 21/47; G01N 33/50

(52) U.S. Cl. CPC G01N 21/47; G01N 33/5005; G01N 2201/06113

(57) ABSTRACT

Approximations to distinguish between X sperm and Y sperm are obtained by detecting light scattered into a pre-set angular range. The scattered light is scattered in turn by nuclei of sperm moving single-file in a sample container. The sperm are not modified prior to the light scattering and are not modified by the light scattering. Approximations are improved by at least a second detection of scattered light.

This application claims priority of U.S. provisional application 61/737,808 filed 16 Dec. 2012 which is incorporated in full herein by reference.

### SUMMARY

It is a new result and unexpected discovery that an approximation that a sperm is an X sperm (X chromosome containing) rather than an Y sperm (Y chromosome containing) can be obtained by detecting light scattered by the sperm into a preset angular range.

It is a new result and unexpected discovery that the approximation that the sperm is an X sperm rather than an Y sperm can be improved by detecting second light scattered by the sperm into a second preset angular range.

This can be done in-turn for each sperm in a single-file flow of sperm. Sperm likely to be X sperm according the approximation can be routed to an X sperm flow and sperm likely to be Y sperm according to the approximation can be routed to a Y sperm flow. Alternatively, unwanted sperm can be destroyed in the flow.

Sperm are not modified prior to the light scattering and are not modified by the light scattering.

### BRIEF DESCRIPTION OF THE DRAWINGS

FIG. 1 schematically illustrates elements of the system.

FIG. 2 shows calibration data with the approximate angle of an X-nucleus peak angle of incident light scattered by an X sperm nucleus 31 and with the approximate angle of a Y-nucleus peak angle of incident light scattered by a Y sperm nucleus 41.

### DETAILED DESCRIPTION

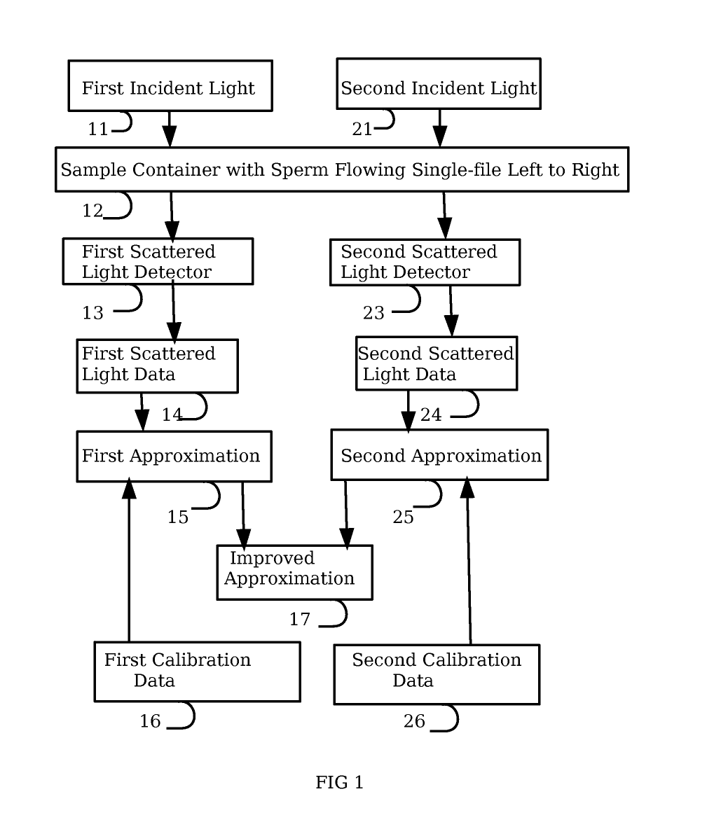

Elements of a system to distinguish X sperm from Y sperm are illustrated schematically in FIG. 1.

The system comprises a first source of first incident light 11. The first incident light has a first incident light central ray. The first incident light has a first incident light wavelength. A 780 nm laser provides good data.

The system also comprises a sample container 12 to contain a test sperm from a sample of sperm. The test sperm and other sperm from the sample of sperm flow single-file along the sample container.

The sample container bore is just large enough for one sperm. The test sperm can be moved by a micro-fluidic actuator to a first detection part of the sample container and then moved on after detection.

The sample container is traversed by the first incident light central ray at the first detection region.

The system also comprises a first scattered light detector 13. The first scattered light detector is positioned to detect first incident light scattered by the nucleus of the test sperm in the sample container. A Logitech™ 160 webcam provides good data with a single 1/30 second exposure.

The system also comprises a first detection by the first scattered light detector of first scattered light data 14 (DSL₁).

The first scattered light data (DSL₁) comprise first incident light scattered by the nucleus of the test sperm into a first preset angular range away from the first incident light central ray.

There can be a preset intensity threshold for this detection to insure that the test sperm is scattering the first incident light.

Parts of the first incident light scattered beyond the first preset angular range can be sampled by the first scattered light detector to insure that the scattering is by the test sperm rather than by some debris flowing along the sample container.

The first scattered light data (DSL₁) comprises a first nucleus scattering peak angle (TP₁) away from the first incident light central ray of first incident light scattered by the test sperm nucleus.

The first X-nucleus and Y-nucleus peak angles can be defined various ways. One example is the angle away from the first incident light central ray of the greatest intensity of the peak.

The system also comprises first calibration data 16 (CSL₁).

The first calibration data comprise a first approximate Y-nucleus peak angle 41 (CY₁) away from the first incident light central ray of first incident light scattered by nuclei of Y sperm from the sample of sperm.

The first calibration data also comprises a first approximate X-nucleus peak angle 31 (CX₁) away from the first incident light central ray of first incident light scattered by nuclei of X sperm from the sample of sperm.

At least CX₁ and CY₁ are included in the first preset angular range.

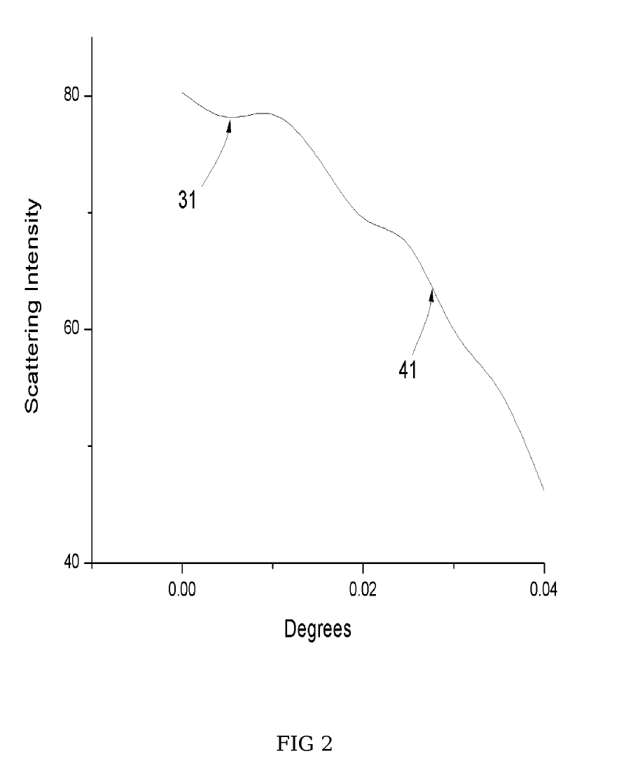

FIG. 2 shows typical first calibration data obtained by detecting with the first scattered light detector first incident light scattered in-turn by five thousand unsorted sperm flowing single-file in the sample container. CX₁ can be set just before the X sperm peak as indicated 31 and CY₁ can be set just after the Y sperm peak as indicated 41.

The system also comprises a first comparison of first scattered light data (DSL₁) with first calibration data (CSL₁) to obtain a first approximation 15 (A₁) that the test sperm is an X sperm rather than an Y sperm:

A₁=f₁(CSL₁,DSL₁).

In a multiple detection option the system can also comprise a second source of second incident light 21.

A multiple detection option can comprise positioning the test sperm in the first detection part of the sample container and re-positioning the test sperm in the detection part of the sample container by a micro-fluidic actuator. Other ways to vary orientation of the sperm relative to incident light and scattered light detection can be used.

The second incident light has a second incident light central ray which intersects the sample container 12.

The second incident light is positioned so that the test sperm nucleus scatters second incident light after the test sperm nucleus scatters first incident light.

The second incident light has a second incident light wavelength.

In an option the second wavelength can be not equal to the first wavelength. In an option the second wavelength can be equal to the first wavelength.

In this option the system can also comprise a second scattered light detector 23. The second scattered light detector is positioned to detect second incident light scattered by the test sperm nucleus.

The second scattered light detector and the first scattered light detector can be parts of one scattered light detector.

In the multiple detection option the system can also comprise a second detection by the second scattered light detector of second scattered light data 24 (DSL₂).

The second scattered light data (DSL₂) comprises second incident light scattered by the nucleus of the test sperm into a second preset angular range away from the second incident light central ray.

The second scattered light data (DSL₂) comprises a second test sperm nucleus scattering peak angle (TP₂) away from the second incident light central ray of second incident light scattered by the test sperm nucleus.

In this option the system also comprises second calibration data 26 (CSL₂).

The second calibration data comprise a second approximate Y-nucleus peak angle (CY₂) away from the second incident light central ray of second incident light scattered by nuclei of Y sperm from the sample of sperm.

The second calibration data also comprises a second approximate X-nucleus peak angle (CX₂) away from the second incident light central ray of second incident light scattered by nuclei of X sperm from the sample of sperm.

At least CX₂ and CY₂ are included in the second preset angular range.

In this option the system can also comprise a second comparison of second scattered light data (DSL₂) with second calibration data (CSL₂) to obtain a second approximation 25 (A₂) that the test sperm is an X sperm rather than a Y sperm:

A₂=f₂(CSL₂,DSL₂).

In this option the system also comprises an improved approximation 17 (A₂′) that the sperm is an X sperm rather than a Y sperm obtained using:

A₂′=f′(A₁,A₂).

In an option the equation

A₁=f₁(CSL₁,DSL₁) is

A₁=(CY₁−TP₁)/(CY₁−CX₁).

When the approximation A₁ is greater than 0.5 it is likely that the test sperm is an X sperm rather than a Y sperm. When A₁ is less than 0.5 it is likely that the test sperm is a Y sperm rather than an X sperm.

In an option the equation

A₂=f₂(CSL₂,DSL₂) is

A₂=(CY₂−TY₂)/(CY₂−CX₂); and the equation

A₂′=f′(A₁,A₂) is

A₂′=(A₁*A₂)/[(A₁*A₂)+(1−A₁)*(1−A₂)].

Sensitivity and reliability of this approximation and the probability that the approximation does distinguish an X sperm from a Y sperm can all be improved by increasing the number of detections of test sperm nucleus scattering peak angles (TPᵢ) for the test sperm and of the approximations (Aᵢ) from each of the detections.

For n values of Aᵢ having an average value A_{avg} greater than 0.5, with i being the number of values of Aᵢ greater than A_{avg}, with r=|n−i|, with x=i−r+1, and with y=n−i−r, then the probability P that the sperm is an X sperm can be approximated by:

P=A_{avg}̂x*(1−A_{avg})̂y/[A_{avg}̂x*(1−A_{avg})̂y+A_{avg}̂y*(1−A_{avg})̂x].

Many sperm can flow single-file along the sample container so that each in-turn is a test sperm and the system can approximate for each that it is an X sperm rather than a Y sperm, and alternatively that it is a Y sperm rather than an X sperm.

Sperm likely to be X sperm according the approximation can be routed to an X sperm flow and sperm likely to be Y sperm according to the approximation can be routed to a Y sperm flow. Alternatively, unwanted sperm can be destroyed in the flow.

In an option the system can also comprise a first calibration detection by the first scattered light detector of the first calibration data (CSL₁).

The first calibration detection comprises a first average of first sperm scattered light scattered in-turn by nuclei of each member of a first plurality of un-sorted sperm from the sample of sperm flowing single-file, in the sample container.

In an option, the multiple detection option system can comprise a second calibration detection by the second scattered light detector of second calibration data (CSL₂).

The second calibration data comprises a second average of second sperm scattered light scattered in-turn by nuclei of each member of a second plurality of un-sorted sperm from the sample of sperm flowing single-file in the sample container.

First calibration data depend on the configuration of the system and depend on differences in samples of sperm. Second calibration data depend on the configuration of the system and depend on the same differences in samples of sperm which effect first calibration data. If the system is configured such that second calibration data is redundant, then first calibration data can be used in place of second calibration data.

FIG. 2 shows the result of a first calibration detection. Scattered light intensities were detected in-turn by the first scattered light detector for five thousand unsorted sperm flowing single-file along the sample container. Reliability and sensitivity of locations of CX₁ and CY₁ and thus of the system can be improved by detecting ten times this number of sperm for the calibration data.

CX₁ and CX₂ can be set just before the approximate X-nuclei peaks. CY₁ and CY₂ can be set just after the approximate Y-nuclei peaks.

Claimed is:

1. A system to distinguish an X sperm from a Y sperm, the system comprising:
1.1. a first source of first incident light,
1.1.1. the first incident light having a first incident light central ray,
1.1.2. the first incident light having a first incident light wavelength;
1.2. a sample container to contain a test sperm from a sample of sperm;
1.2.1. the test sperm and other sperm from the sample of sperm flowing single-file along the sample container,
1.2.2. the test sperm having a nucleus,
1.2.3. the sample container being traversed by the first incident light central ray,
1.3. a first scattered light detector,
1.3.1. the first scattered light detector positioned to detect first incident light scattered by the nucleus of the test sperm in the sample container;
1.4. a first detection by the first scattered light detector of first scattered light data (DSL₁),
1.4.1. the first scattered light data (DSL₁) being first incident light scattered by the nucleus of the test sperm into a first preset angular range away from the first incident light central ray,
1.4.2. the first scattered light data (DSL₁) comprising a first nucleus scattering peak angle (TP₁) away from the first incident light central ray of first incident light scattered by the test sperm nucleus;
1.5. first calibration data (CSL₁) comprising:
1.5.1. a first approximate Y-nucleus peak angle (CY₁) away from the first light central ray of first incident light scattered in-turn by nuclei of Y sperm in the sample of sperm flowing single-file along the sample container,
1.5.2. a first approximate X-nucleus peak angle (CX₁) away from the first light central ray of first incident light scattered in-turn by nuclei of X sperm in the sample of sperm flowing single-file along the sample container,
1.5.3. at least CX₁ and CY₁ being included in the first preset angular range;
1.6. a first comparison of first scattered light data (DSL₁) with first calibration data (CSL₁) to obtain a first approximation (A₁) that the test sperm is an X sperm rather than an Y sperm:
A₁=f₁(CSL₁,DSL₁).

2. The system of claim 1 also comprising:
2.1. a second source of second incident light,
2.1.1. the second incident light having a second incident light wavelength,
2.1.2. the second incident light having a second incident light central ray,
2.1.3. the second incident light being positioned so that the test sperm nucleus scatters second incident light after the test sperm nucleus scatters first incident light;
2.2. a second scattered light detector,
2.2.1. the second scattered light detector being positioned to detect second incident light scattered by the nucleus of the test sperm in the sample container;
2.3. a second detection by the second scattered light detector of second scattered light data (DSL₂),
2.3.1. the second scattered light data (DSL₂) comprising second incident light scattered by the nucleus of the test sperm into a second preset angular range away from the second incident light central ray,
2.3.2. the second scattered light data (DSL₂) comprising a second nucleus scattering peak angle (TP₂) away from the second incident light central ray of second incident light scattered by the test sperm nucleus;
2.4. second calibration data (CSL₂) comprising:
2.4.1. a second approximate Y-nucleus peak angle (CY₂) away from the second light central ray of second incident light scattered in-turn by nuclei of Y sperm from the sample of sperm flowing single-file along the sample container,
2.4.2. a second approximate X-nucleus peak angle (CX₂) away from the second light central ray of second incident light scattered in-turn by nuclei of X sperm from the sample of sperm flowing single-file along the sample container,
2.4.3. at least CX₂ and CY₂ being included in the second preset angular range;
2.5. a second comparison of second scattered light data (DSL₂) with second calibration data (CSL₂) to obtain a second approximation (A₂) that the test sperm is an X sperm rather than an Y sperm:
A₂=f₂(CSL₂,DSL₂); and
2.6. an improved approximation (A₂′) that the sperm is an X sperm rather than a Y sperm obtained using:
A₂′=f′(A₁,A₂).

3. The system of claim 2 with the added limitation that the second incident light wavelength is not equal to the first incident light wavelength.

4. The system of claim 1 with the added limitations that the equation
A₁=f₁(CSL₁,DSL₁) is
A₁=(CY₁−TP₁)/(CY₁−CX₁).

5. The system of claim 2 with the added limitations that the equation
A₂=f₂(CSL₂,DSL₂) is
A₂=(CY₂−TP₂)/(CY₂−CX₂); and the equation
A₂′=f′(A₁,A₂) is
A₂′=(A₁*A₂)/[(A₁*A₂)+(1−A₁)*(1−A₂)].

6. The system of claim 1 with the added limitation of a first calibration detection by the first scattered light detector of first calibration data (CSL₁),
6.1. the first calibration detection comprising a first average of first incident light scattered in-turn by nuclei of each member of a first plurality of un-sorted sperm from the sample of sperm flowing single-file in the sample container.

7. The system of claim 2 with the added limitation of a second calibration detection by the second scattered light detector of second calibration data (CSL₂),
7.1. the second calibration detection comprising a second average of second incident light scattered in-turn by nuclei of each member of a second plurality of un-sorted sperm from the sample of sperm flowing single-file in the sample container.

8. A system to distinguish an X sperm from a Y sperm, the system comprising:
8.1. a first source of first incident light,
8.1.1. the first incident light having a first incident light central ray,
8.1.2. the first incident light having a first incident light wavelength;
8.2. a sample container to contain a test sperm from a sample of sperm;
8.2.1. the test sperm and other sperm from the sample of sperm flowing single-file along the sample container,
8.2.2. the test sperm having a nucleus, and
8.2.3. the sample container being traversed by the first incident light central ray,
8.3. a first scattered light detector,
8.3.1. the first scattered light detector positioned to detect first incident light scattered by the nucleus of the test sperm in the sample container;
8.4. a first detection by the first scattered light detector of first scattered light data (DSL₁),
8.4.1. the first scattered light data (DSL₁) being first incident light scattered by the nucleus of the test sperm into a first preset angular range away from the first incident light central ray,
8.4.2. the first scattered light data (DSL₁) comprising a first nucleus scattering peak angle (TP₁) away from the first incident light central ray of first incident light scattered by the test sperm nucleus;
8.5. first calibration data (CSL₁) comprising:
8.5.1. a first approximate Y-nucleus peak angle (CY₁) away from the first light central ray of first incident light scattered by nuclei of Y sperm in the sample of sperm flowing single-file along the sample container,
8.5.2. a first approximate X-nucleus peak angle (CX₁) away from the first light central ray of first incident light scattered by nuclei of X sperm in the sample of sperm flowing single-file along the sample container,
8.5.3. at least CX₁ and CY₂ being included in the first preset angular range;
8.6. a first comparison of first scattered light data (DSL₁) with first calibration data (CSL₁) to obtain a first approximation (A₁) that the test sperm is an X sperm rather than an Y sperm:
A₁=f₁(CSL₁,DSL₁);
8.7. a second source of second incident light,
8.7.1. the second incident light having a second incident light wavelength,
8.7.2. the second incident light having a second incident light central ray,
8.7.3. the second incident light being positioned so that the test sperm nucleus scatters second incident light after the test sperm nucleus scatters first incident light;
8.8. a second scattered light detector,
8.8.1. the second scattered light detector being positioned to detect second incident light scattered by the nucleus of the test sperm in the sample container;
8.9. a second detection by the second scattered light detector of second scattered light data (DSL₂),
8.9.1. the second scattered light data (DSL₂) comprising second incident light scattered by the nucleus of the test sperm into a second preset angular range away from the second incident light central ray,
8.9.2. the second scattered light data (DSL₂) comprising a second nucleus scattering peak angle (TP₂) away from the second incident light central ray of second incident light scattered by the test sperm nucleus;
8.10. second calibration data (CSL₂) comprising:
8.10.1. a second approximate Y-nucleus peak angle (CY₂) away from the second light central ray of second incident light scattered in-turn by nuclei of Y sperm from the sample of sperm flowing single-file along the sample container,
8.10.2. a second approximate X-nucleus peak angle (CX₂) away from the second light central ray of second incident light scattered in-turn by nuclei of X sperm from the sample of sperm flowing single-file along the sample container,
8.10.3. at least CX₂ and CY₂ being included in the second preset angular range;
8.11. a second comparison of second scattered light data (DSL₂) with second calibration data (CSL₂) to obtain a second approximation (A₂) that the test sperm is an X sperm rather than an Y sperm:
A₂=f₂(CSL₂,DSL₂); and
8.12. an improved approximation (A₂′) that the sperm is an X sperm rather than a Y sperm obtained using:
A₂′=f′(A₁,A₂).

9. The system of claim 8 with the added limitation that the second incident light wavelength is not equal to the first incident light wavelength.

10. The system of claim 8 with the added limitations that the equation
A₁=f₁(CSL₁,DSL₁) is
A₁=(CY₁−TP₁)/(CY₁−CX_{R}) and the equation
A₂=f₂(CSL₂,DSL₂) is
A₂=(CY₂−TP₂)/(CY₂−CX₂) and the equation
A₂′=f′(A₁,A₂) is
A₂′=(A₁*A₂)/[(A₁*A₂)+(1−A₁)*(1−A₂)].

11. The system of claim 8 with the added limitation of a first calibration detection by the first scattered light detector of first calibration data (CSL₁),
11.1. the first calibration detection comprising a first average of first incident light scattered in-turn by nuclei of each member of a first plurality of un-sorted sperm from the sample of sperm flowing single-file in the sample container.

12. The system of claim 8 with the added limitation of a second calibration detection by the second scattered light detector of second calibration data (CSL₂),
12.1. the second calibration detection comprising a second average of second incident light scattered in-turn by nuclei of each member of a second plurality of un-sorted sperm from the sample of sperm flowing single-file in the sample container.
